(19) United States
(12) Patent Application Publication
(10) Pub. No.: US 2008/0208105 A1
(43) Pub. Date: Aug. 28, 2008

(54) LASER ENERGY DEVICE FOR SOFT TISSUE REMOVAL

(76) Inventors: Brian D. Zelickson, Minneapolis, MN (US); Thomas Dressel, Bloomington, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/955,128

(22) Filed: Dec. 12, 2007

Related U.S. Application Data
(60) Provisional application No. 60/869,581, filed on Dec. 12, 2006.

(51) Int. Cl. A61B 18/20; A61M 1/00

(52) U.S. Cl. 604/20; 604/22

(57) ABSTRACT

This invention relates to a device and method for improving the surgical procedure of soft tissue removal by aspiration and more particularly to a device and method utilizing laser energy directed substantially across the inlet port to more readily and safely facilitate the separating of soft tissue from a patient in vivo. This invention has immediate and direct application to the surgical procedure of liposuction or body contouring as well as application in the surgical procedures of other soft tissue removal such as brain tissue, eye tissue, and other soft tissue inaccessible to other soft tissue aspiration techniques.

### FIELD OF THE INVENTION

This invention relates to a device and method for improving the surgical procedure of soft tissue removal by aspiration and more particularly to a device and method utilizing laser energy directed at the edge of the inlet port to more readily and safely facilitate the separating of soft tissue from a patient in vivo. This invention has immediate and direct application to the surgical procedure of liposuction or body contouring as well as application in the surgical procedures of other soft tissue removal such as brain tissue, eye tissue, and other soft tissue.

### BACKGROUND OF THE INVENTION

Within the past decade, the surgical use of lasers to cut, cauterize and ablate tissue has been developing rapidly. Advantages to the surgical use of laser energy lie in increased precision and maneuverability over conventional techniques. Additional benefits include prompt healing with less post-operative pain, bruising, and swelling. Lasers have become increasingly important, especially in the fields of Ophthalmology, Gynecology, Plastic Surgery and Dermatology, as a less invasive, more effective surgical therapeutic modality which allows the reduction of the cost of procedures and patient recovery times due to diminished tissue trauma, bleeding, swelling and pain. The CO₂ laser has achieved wide spread use in surgery for cutting and vaporizing soft tissue. The CO₂ laser energy has a very short depth of penetration, however, and does not effectively cauterize small blood vessels. Other means such as electrocautery must be used to control and minimize blood loss. Infrared lasers, such as the Neodymium-YAG laser, on the other hand, because of its greater depth of tissue penetration, is very effective in vaporizing soft tissue and cauterizing small blood vessels. But as a result of this great depth of tissue penetration, infrared lasers, such as the Neodymium-YAG laser, have achieved limited use in the field of soft tissue surgery because of the possibility of unwanted damage to deeper tissues in the path of the laser energy beam. Recently, some infrared wavelength have been shown to have selectivity to lipids and adipose tissue. The potential benefit of these wavelengths it that they can selectively melt or destroy fat with less energy while sparing other surrounding tissues such as nerves and collagen. Various visible light lasers have shorter wavelengths and therefore do not penetrate deeply into tissue, while having the benefit of being able to selectively target structures such as blood vessels to help control bleeding.

Liposuction, a surgical technique of removing unwanted fat deposits for the purpose of body contouring, has achieved widespread use. In the U.S., over 400,000 liposuction procedures were performed in 2005 alone. This technique utilizes a hollow tube or cannula with a blunt tip and a side hole or tissue aspiration inlet port near its distal end. The proximal end of the cannula has a handle and a tissue outlet port connected to a vacuum aspiration pump. In use, a small incision is made, the cannula tip and adjacent tissue inlet port is passed beneath the surface of the skin into the unwanted fat deposit. The vacuum pump is then activated drawing a small amount of tissue into the lumen of the cannula via the inlet port. Longitudinal motion of the cannula then removes the unwanted fat by a combination of sucking and ripping actions. This ripping action causes excessive trauma to the fatty tissues resulting in considerable blood loss and post-operative bruising, swelling and pain. Proposed advances in the techniques and apparatus in this field have been primarily directed to the design of the aspiration cannula, and more recently have involved the application of ultrasound and irrigation to melt and solubilize fatty tissue or the use of an auger, within the lumen of the cannula, to facilitate soft tissue removal. These proposed advances do not adequately address the goals of the surgical procedure: the efficient and precise removal of soft tissue with minimal tissue trauma and blood loss.

Other laser energy devices have been developed that are a modification of a suction lipectomy cannula and have already been clinically used. Such devices position soft tissue within a protective chamber, allowing a Neodymium-YAG laser energy beam to cut and cauterize the soft tissue without fear of unwanted damage to surrounding or deeper tissues. Such devices allow the removal of soft tissue while minimizing tissue trauma by eliminating the ripping action inherent in the conventional liposuction method. Furthermore, such devices, by eliminating the ripping action of the conventional liposuction method, expand the scope of soft tissue removal. These earlier methods were limited by the fact that the interior positioning of the Nd:YAG laser fiber caused a decrease in the cross sectional area of the lumen and thus clogging and decreased efficiency. Another drawback if the design was the fact that the Nd:YAG laser fiber was positioned proximal to the opening of the liposuction catheter. Thus, all the suctioned fat would be sucked directly into the firing end of the fiber causing charring and destruction of the laser fiber tip. Further limitation of the earlier invention was the fact that the disclosure was limited to the using a single wavelength Nd:YAG laser. This did not enable one to selectively target specific structures such as fat and blood vessels and also made it necessary to enclose the fiber to minimize injury to surrounding vital structures. Generally, the liposuction method is limited to the aspiration of fat. Other soft tissues, such as breast tissue, lymphangiomas, and hemangiomas are too dense or too vascular to allow efficient and safe removal utilizing the liposuction method. The laser energy devices utilize a precise cutting and coagulating action of the laser or other fiber delivered cautery and coagulating laser, within the cannula, thereby permitting the removal of these dense or vascular soft tissues.

Additionally the laser energy devices described above, by controlling the depth of penetration of the laser energy either within the protective aspiration cannula, or with focusing the beam or using different spot sizes and or wavelengths, expands the surgical applicability. This laser can be used, for example, in the precise removal of brain tissue without fear of unwanted damage to surrounding or deeper tissues. Furthermore, the CO₂ laser is extensively used for the vaporization of brain tumors, but because of its inability to effectively coagulate blood vessels, other methods such as electrocautery must be used to control blood loss during the procedure. In addition, because the vaporization of tissue generates large volumes of noxious and potentially toxic smoke, expensive, noisy and cumbersome suction devices must be used to eliminate the smoke from the surgical field. However, the laser energy devices, by utilizing the more effective coagulating power of visible and infrared lasers, permits the combined action of tissue cutting, control of blood loss, and elimination of smoke from the surgical field.

### BRIEF DESCRIPTION OF THE INVENTION

While the laser energy devices described above have provided many beneficial characteristics and attributes, the chance of occlusion of the cannula has been identified as a potential issue due to the laser fiber guide tube being located totally or partially inside the lumen of the cannula. The inclusion of the laser fiber guide tube inside the cannula results in a decreased cross sectional area within the cannula and thereby a higher potential for occlusion and decreased efficiency. The location of the tip of the laser fiber also increases the likelihood of the aspirated soft tissue coming into direct contact with the laser fiber tip resulting in fiber charring. In general, the devices of the present invention can include many of the same or similar components as the laser energy devices described above. Embodiments of such devices, components and their methods of manufacture and use are disclosed and/or suggested in U.S. Pat. Nos. 4,985,027 and 5,102,410, the contents of which are incorporated by reference herein. However, in various embodiments of the present invention the laser guide tube is located inside the handle at the proximal end of the cannula, but it is located outside of the lumen of the cannula and extends along the length of the cannula to the distal end. In such embodiments, the laser guide tube is positioned near the proximal end of the inlet port at the distal end of the cannula and can be curved inward to allow the laser fiber to direct the laser energy across or slightly into the inlet port. In other embodiments of the present invention the laser fiber enters the cannula but reflects the energy off of a reflective surface, such as a mirror, positioned at the far distal end of the cannula thereby allowing the reflected laser energy to be directed across the inlet port, at the port or outside the port. The geometry of the reflecting surface can be altered to allow for focusing or defocusing the reflected laser energy at near or outside the inlet port. Thus in practice the cannula of these embodiments operate in essentially the same way as in the above described laser energy devices, but without the potential disadvantage of having the laser guide tube within the lumen. In addition, by positioning the laser fiber tip safely out of the soft tissue stream this new laser guide tube design greatly reduces the possibility of laser fiber charring and damage.

Further embodiments include: the use of different or multiple wavelengths, spot sizes and focusing means in order to selectively target specific tissues and/or localize the depth of the laser penetration; and, adding multiple aspiration ports on the cannula to enhance tissue removal.

It is noted that the basic design of the present invention can be also scaled down to permit soft tissue aspiration in other parts of the body. For example, an appropriately sized version of the present device can be used for safe removal of scar tissue from within the eye or adjacent to the retina and lens tissue from within the eye. Other appropriately sized and scaled versions of the present device can also be used for the removal of other unwanted soft tissues within the body. For example: removal of unwanted tracheal tissue, such as bronchial adenomas; removal of polyps and other soft tissue from within the lumen of the gastrointestinal tract and nasal cavity; for endometrial ablations within the uterus; in conjunction with laparoscopic techniques to remove endometrial tissue within the abdomen.

Various embodiments of the present invention provides a soft tissue aspiration device comprising an aspiration cannula and a laser guide tube extending longitudinally along the exterior of the cannula. In such embodiments, the guide tube houses a laser energy transmission guide for conducting the laser energy to the soft tissue removal site within the patient's body and also housing a fluid flow path around the laser energy transmission guide. The aspiration cannula has a proximal and a distal end. The cannula is provided with a soft tissue aspiration inlet port adjacent to the cannula distal end. The proximal end of the cannula is attached to a handle which is provided with a fluid flow delivery port, a laser energy transmission guide inlet port, and an aspirated soft tissue outlet port. The fluid and laser fiber guide tube extends longitudinally from near the proximal end of the soft tissue aspiration device, along the exterior wall of the cannula, to a point near the inlet port, then curves inward so as to direct laser energy, within the cannula, across the aspiration inlet port. A laser energy transmission guide extends from a laser energy source to the proximal end of the handle and longitudinally within the guide tube to a point immediately prior to the terminal point of the guide tube. In various embodiments, within the soft tissue aspiration device laser guide tube, the laser energy transmission guide is surrounded by fluid flow from a fluid source to the laser guide tube terminal point. However, with some of the embodiments of the present invention it is clear that one could use the device safely without a fluid source, without injuring the fiber tip.

This invention also provides a surgical method of aspirating soft tissue from a patient in vivo using the device just described.

### BRIEF DESCRIPTION OF THE DRAWINGS

FIG. 1 is a side cut-away elevation view of a soft tissue aspiration device of the present invention.

FIG. 2 is a side cut-away elevation view of a soft tissue aspiration device of the present invention including a mirror positioned at the cannula tip.

FIG. 3 is a partial exploded side cut-away elevation view, showing the distal end of the laser fiber guide adjacent the soft tissue aspiration inlet port.

FIG. 4 is a partial longitudinal section view of the handle and proximal end cap suitable for use with embodiments of the device showing the attachments of the fluid and laser guide tube to the laser fiber and sources of fluid.

FIG. 5 is a partial longitudinal section view of a handle suitable for use with embodiments of the device showing the fluid and laser fiber guide tube, Teflon coaxial fluid delivery tube and channel, and laser energy transmission guide.

FIG. 6 is a partial exploded longitudinal section of a laser fiber optic delivery system with Teflon coaxial fluid delivery tube.

FIG. 7 is a partial exploded longitudinal section view of a handle and proximal end cap suitable for use with embodiments of the device showing the attachments of the laser energy transmission guide to the alternative fiber optic delivery system and alternative fluid source.

FIG. 8 is a partial exploded longitudinal section of an alternative laser energy transmission guide without Teflon coaxial fluid delivery tube.

FIG. 9 is a cut-away detail of the first laser soft tissue device illustrated in position for performing liposuction within a fatty deposit of a body intermediate overlying epidermal layer and underlying muscle layer.

FIG. 10 is a partial longitudinal section of the distal end of a laser guide tube including a cannula and laser energy transmission guide within, according to one embodiment of the invention.

FIG. 11 is a partial exploded perspective view of the distal end of a laser guide tube including a cannula and laser energy transmission guide within, according to one embodiment of the invention.

FIG. 12 is a partial perspective view of the distal end of a laser guide tube including a cannula and laser energy transmission guide within, according to one embodiment of the invention.

FIG. 13 is a perspective view of the distal end of an aspiration device according one embodiment of the invention having aspiration inlet cap remove to expose an unsealed laser guide lumen.

FIG. 14 is a perspective view of the distal end of an aspiration device according one embodiment of the invention having aspiration inlet cap remove to expose an epoxy-sealed laser guide lumen.

FIG. 15 is a partial longitudinal section of the distal end of a cannula including a laser energy focusing device and a reflective surface according to one embodiment of the invention.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the components, principles and practices of the present invention.

FIGS. 1, 2 and 10 depict embodiments of a laser soft tissue aspiration device 100 wherein the device comprises an aspiration cannula 112, a laser guide tube 36, an aspiration inlet port 20, and a laser energy transmission guide 115. The aspiration cannula 112 includes a lumen 113 providing for fluid and/or soft tissue flow within the cannula 112. The lumen 113 is in communication with one or more aspiration inlet ports 20 at a distal end 114 of the aspiration cannula 112. An aspirated soft tissue outlet port 28 at a proximal end 116 of the device 100 and in fluid flow connection to the lumen 1 3can couple an aspiration source (not shown) with the lumen 113. The laser guide tube 36 extends longitudinally along the exterior of the cannula 112 to a termination point 40 proximal the aspiration inlet port(s) 20. Within the laser guide tube 36 and external to the cannula 112, a laser energy transmission guide 115 extends from a laser energy source (not shown) to the termination point 40 at the distal end 114 of the cannula 112. The distal end 56 of the laser energy transmission guide 115 can be configured to direct laser energy across the face of the aspiration inlet port(s) 20 such that the laser energy remains within the lumen 113. In various embodiments, the laser soft tissue aspiration device 100 can include a handle 22 at the proximal end 116 of the aspiration cannula 112.

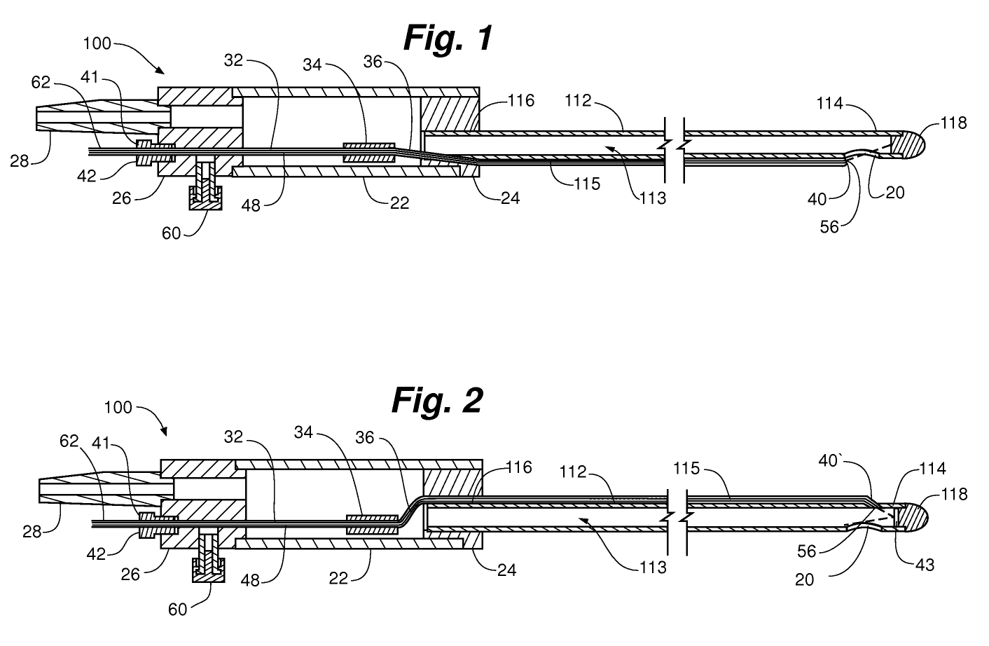

The device 100 in the embodiments of FIGS. 1 and 2, includes an aspiration cannula 112 having one or more soft tissue inlet port(s) 20 adjacent to the distal end 114 and cannula tip 118. A handle 22 retains distal handle end cap 24 and proximal handle end cap 26. The distal handle end cap 24 retains the cannula proximal end 116 and a laser guide tube 36. The proximal handle end cap 26 retains the aspirated soft tissue outlet port 28, and a fluid and fiber guide tube system. The soft tissue outlet port 28 can be connected to an aspiration source by a plastic tubing (not shown).

It will be apparent to those skilled in this art, that aspiration cannula 112 dimensions can vary for different applications. For example, a shorter and thinner aspiration cannula 112 can be useful for procedures involving more restricted areas of the body, such as under the chin and around small appendages. A longer and larger diameter cannula can be useful in areas such as the thighs and buttocks where the cannula can be extended into soft tissue over a more extensive area. The length of the laser guide tube 36 is determined by the length of the soft tissue aspiration cannula 112. In various embodiments, aspiration cannulas are made from stainless steel and can be configured in a variety of different lengths. In various embodiments of the present invention, aspiration cannula cross-sectional dimensions include: 0.312″ O.D.×0.016″ wall (0.280″ inner diameter (“I.D.”)), 0.250″ O.D.×0.016″ wall (0.218 ″ I.D.), 0.188″ O.D.×0.016″ wall (0.156″ I.D.), and 0.156″ O.D.×0.016″ wall (0.124″ I.D.).

As illustrated in FIG. 1, some embodiments of the cannula tip 118 can advantageously be a generally rounded, blunt or bullet shaped tip attached to the cannula 112 by welding or soldering. It is noted that the cannula tip 118 can be replaceable and/or disposable. For example, the cannula tip can include a threaded or snapping means that allows a tip cap (not shown) to screw or snap into the distal end of the cannula. In various embodiments the tip 118 can have a polished reflective interior surface, such as a mirror surface, that can be utilized in various embodiments (see e.g. FIG. 2) to direct the laser energy toward the aspiration inlet port 20. The reflective inner surface of the cap can also be configured to focus or defocus the laser energy depending upon the inner surface geometry. In some embodiments, the cannula tip 118 is made from stainless steel and sized to the same diameter as the aspiration cannula's outer diameter, machined to a blunt tip, and includes a receiving end machined to fit within the cannula's inner diameter.

Numerous variations of the aspiration inlet port(s) 20 are contemplated by the invention. More than one aspiration inlet port 20 can be included in aspiration cannula 112 to provide for more than one location for tissue removal. For example, an embodiment can include two ports spaced at 180 degree intervals, or three inlet ports at 120 degree intervals about a circumference of the distal end of the aspiration cannula 112. In such embodiments one or more laser guide tube(s) 36 and one or more laser energy transmission guide(s) 115 can diverge at a point within handle 22, along the cannula 112 or proximate the tip 118 to direct laser energy across each aspiration inlet port 20. Additionally, aspiration inlet ports can be of any of a variety of shapes (for example oval, circular, squared, angular, parabolic). Additionally, some embodiments can even include a knife (e.g. a quartz or sapphire knife) within or near the aspiration inlet port 20 or tip 118 to mechanically ablate tissue in conjunction with the laser application. However, in various embodiments of the present invention, the edges of the aspiration inlet port(s) 20 are substantially flat or rounded in their cross-section (i.e., not of a sharp nature) such that the ripping action inherent in devices known in the art is avoided.

In various embodiment, such as the embodiment depicted in FIG. 1, one or more laser guide tubes 36 extend longitudinally from the distal handle end cap 24 along the exterior and generally parallel with the aspiration cannula 112 to a termination point 40 immediately proximal to the soft tissue aspiration inlet port 20. Alternatively, other embodiments of the invention, such as that depicted in FIG. 2, can include a laser guide tube 36 that extends longitudinally along the exterior of the aspiration cannula 112 from the proximal handle end cap 26 to a termination point 40′ not immediately proximal to the soft tissue aspiration inlet port 20. For example, the laser guide tube 36 can extend along the cannula 112 and enter on the opposite side of the lumen 113 as the aspiration inlet port 20. In such embodiments, the laser energy can be directed across the aspiration inlet port 20 by a reflective surface 43, such as a mirror. Furthermore, in various embodiments, the entry of the laser guide tube 36 can be positioned on the cannula 112 beyond the position of the aspiration inlet port 20 and closer to the distal end, thereby allowing redirection of the laser energy from the reflective surface 43 while remaining outside the path of fluid or soft tissue flow traveling through the lumen 113 of the cannula 112 during operation of the device 100.

The laser guide tube 36, accommodates a laser energy transmission guide 115 which transmits the laser energy from a laser energy source (not shown) to a terminal point 56 proximate the terminal point of the fluid and laser fiber guide tube 40. An exemplary laser energy transmission guide 115 can be seen in FIG. 8. Such a guide can include a laser fiber sheath 50 encasing laser fiber 54. The sheath 50 and fiber 54 are generally coaxial about longitudinal axis 58, with the sheath terminating at point 52 and laser energy emanating from fiber end 56. In various embodiments of the present invention, the laser fiber sheath 50, is a Teflon laser fiber sheath. Suitable laser fiber 54 materials can include: synthetic laser fibers, glass, quartz, sapphire or other optically transmissible materials.

Fiber end 56 should be positioned proximate laser guide tube end 36, near aspiration inlet port 20. In some embodiments, laser fiber 54 can be curved inward to align fiber end 56 such that the laser energy is directed across and generally toward the internal diameter of aspiration inlet port 20. Additionally, in some embodiments laser fiber 54 can have a cleaved end such that the fiber end 56 is angled relative to (i.e. not parallel with) longitudinal axis 58. In embodiments such as that of FIG. 2, curving or angling of the fiber end 56 can be used to direct the laser energy to properly reflect off of a reflective surface 43, such as a mirror, to cross and extend toward the internal diameter of the inlet port 20.

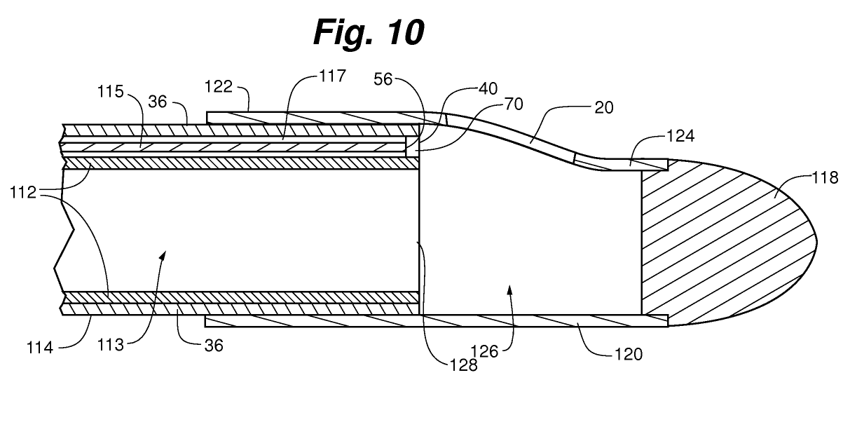

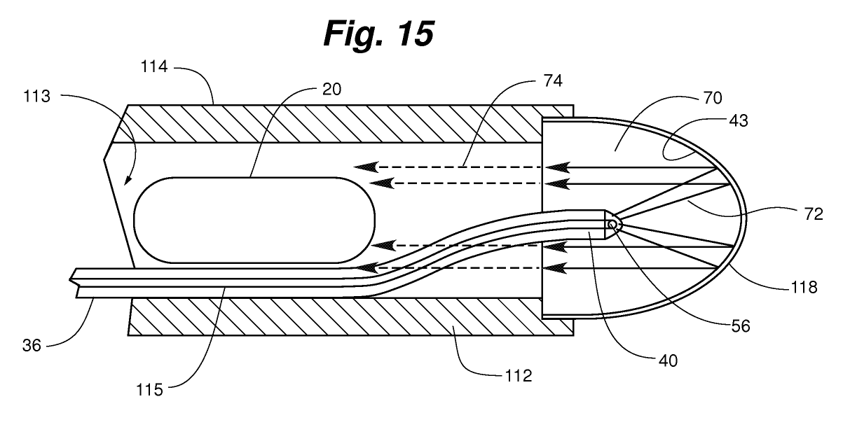

Some embodiments of the present invention include a laser energy diffuser or focusing device 70 (see e.g. FIGS. 10 and 15) interposed between fiber end 56, and aspiration inlet port 20. A diffuser or focusing device 70 can alter the power density of the light impinging on tissue to prevent charring of the laser energy transmission guide 115 and disperse the laser energy across the aspiration inlet port 20. In some embodiments, the fiber end 56 can perpendicularly abut a back face of a laser energy diffuser or focusing device 70 (as shown in FIG. 10). Alternatively, the fiber end 56 can protrude into a diffuser or focusing device 70, or interact at an angle relative to the back face of the diffuser or focusing device 70. A diffuser or focusing device can be constructed of an optical epoxy, thermoplastic (e.g. Lexan), air, glass, or a combination thereof.

FIG. 15 shows a sectional view of the distal end of an aspiration device 114 including a laser energy focusing device 70 disposed within the cannula tip 118, according to some embodiments of the invention. In such embodiments, laser guide tube 36 passing external to lumen 113, can extend distally along the cannula 112 past aspiration inlet port 112. The laser guide tube terminal point 40, can then protrude within the cannula 112, such that the terminal point 56 of laser energy transmission guide 115 can extend into the laser energy focusing device 70 without being exposed to the tissue stream within the lumen 113. In such embodiments, the laser energy focusing device 70 includes a dielectric medium such as, for example, a solid piece of optical epoxy, thermoplastic (e.g. Lexan), air, or glass filling, in tip 118. The tip 118, can include a reflective surface 43 to direct laser energy back toward lumen 113 and aspiration inlet port 20. It is noted that in some embodiments of the present invention, a reflective coating can be administered to the tip 118 to produce the reflective surface 43. In operation, laser energy dispersed from laser energy transmission guide 115, travels through the dielectric medium and off of the reflective surface 43 (denoted by solid lines 72). The energy leaving focusing device 70 (denoted by dashed lines 74), can then ablate tissue entering the aspiration inlet port 20. In such embodiments, laser energy transmission guide 115 and laser guide tube 36 can remain entirely outside the path of fluid or soft tissue flow traveling through the lumen 113. FIG. 15 shows a parabolic shaped device centered on the axis of the lumen 113 focusing light at an infinite distance (i.e., collimating), however reflective surface 43 can assume a number of different shapes (e.g., spherical or elliptical) and positions (e.g., tilted or decentered) to otherwise focus and/or steer light to a finite distance within the lumen 113 and aspiration inlet port 20.

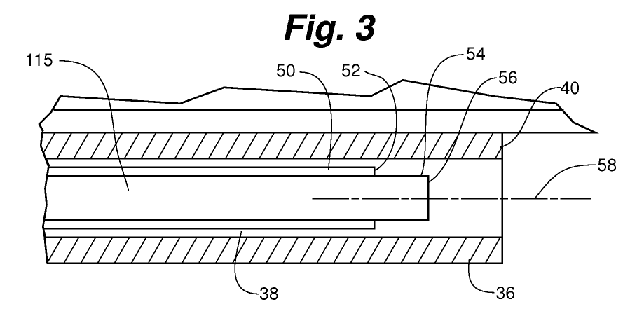

In some embodiments (such as those in FIGS. 1 and 2) the laser guide tube 36 can accommodate a fluid and laser fiber guide tube system. One such laser guide tube 36, is shown in FIG. 3. In this embodiment, the laser guide tube 36 is of sufficient internal diameter to accommodate the laser energy transmission guide 115 (which in this embodiment includes Teflon laser fiber sheath 50 and laser fiber 54) and to provide clearance for a coaxial fluid channel 38. The coaxial fluid channel 38 can provide for fluid cooling of the laser energy transmission guide 115 along its length. In some embodiments, a sensor (not shown) can be positioned within the laser guide tube 36 to indicate whether cooling fluid is passing over the laser energy transmission guide 115 and can function to activate a safety switch, configured to stop laser energy from being transmitted through the laser energy transmission guide, if such cooling is not detected. Such a sensor can be utilized in all embodiments of the present invention, including embodiments wherein a cooling fluid is not utilized to cool the laser energy transmission guide 115.

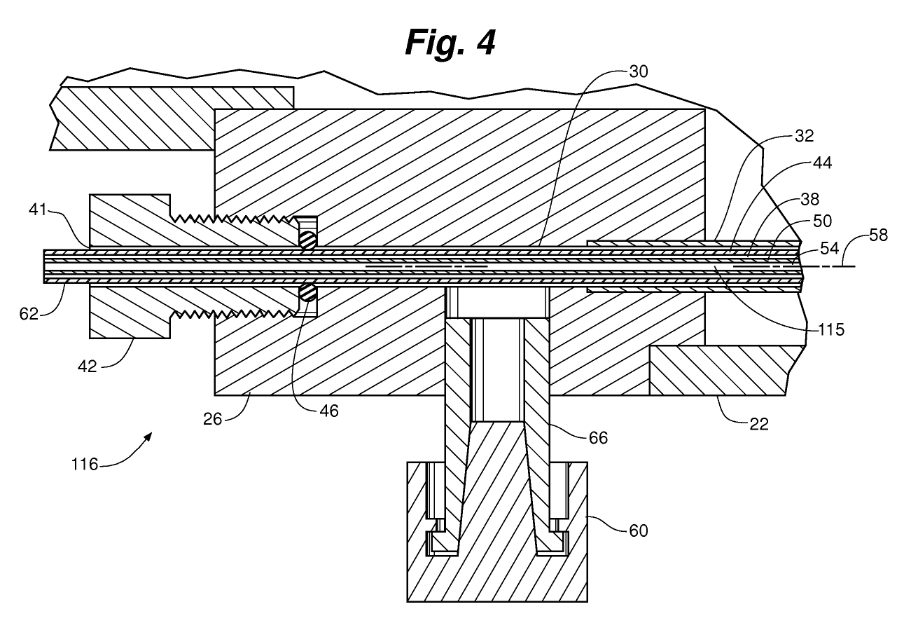

FIG. 4 depicts a proximal end cap 26 coupled to a handle 22 including a fluid and laser fiber guide tube system. Such an embodiment can receive a fluid and laser fiber optic delivery system 62. In the embodiment of FIG. 4, the laser fiber optic system 62 is retained in the handle 22 by a retaining screw 42 and sealed with an O-ring seal 46 at fluid and laser energy source port 41. The fluid and laser fiber optic delivery system 62, can include Teflon coaxial fluid delivery tube 44 and laser energy transmission guide 115. The Teflon coaxial fluid delivery tube 44 is connected to a saline fluid source and pump integral with the laser energy source (not shown) and passes into the proximal end cap of the handle 26, through the fluid and laser guide channel 30 and into the large guide tube 32. Laser energy transmission guide 115 similarly passes through laser guide channel 30 of the proximal end cap 26 and into large guide tube 32. Laser guide channel further includes a connection to optional fluid delivery port 66 fitted with a fluid and air tight plug 60 when the Teflon coaxial fluid delivery tube 44 is used. In these embodiments, the coaxial fluid channel 30 and large guide tube 32 are of sufficient internal diameter to accommodate the Teflon coaxial fluid delivery tube 44.

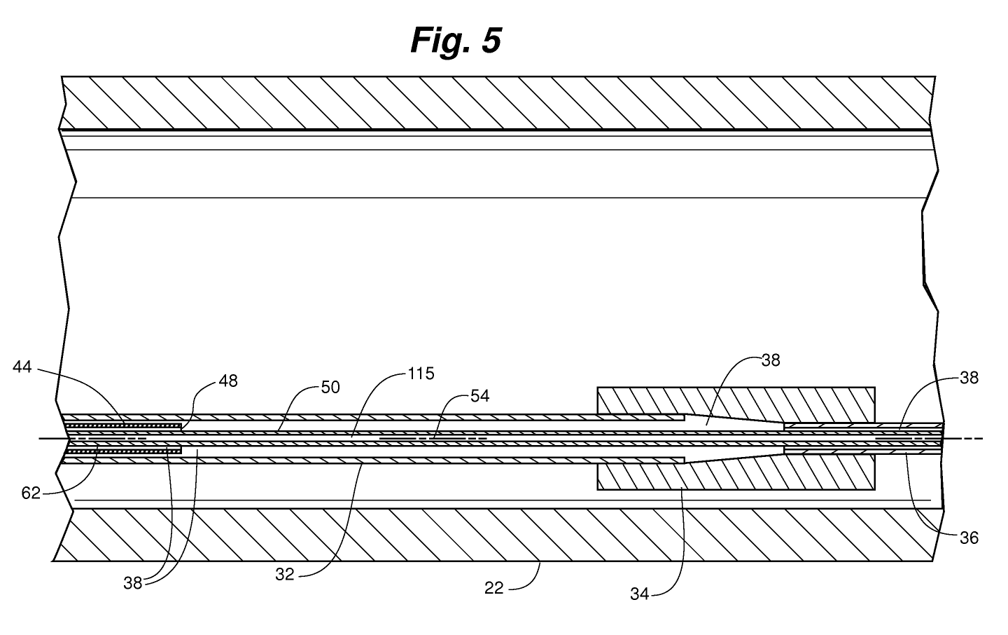

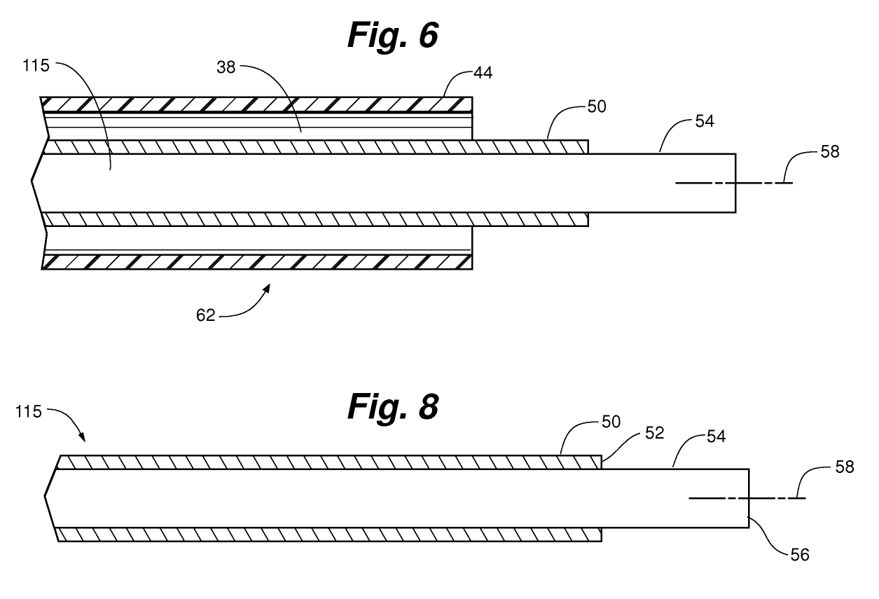

Turning to FIG. 5, other embodiments of the present invention include a large guide tube 32 that proceeds through handle 22 and communicates with a guide tube transition coupler 34. The guide tube transition coupler 34 is positioned within the handle 22 proximal to the proximal end of the cannula 116 and is drilled to accommodate the external diameters of the large guide tube 32 and the laser guide tube 36. Intermediate the proximal end cap 26 and guide tube transition coupler 34 and within the large guide tube 32, the Teflon coaxial fluid delivery tube 44 terminates at point 48. In this manner, the Teflon coaxial fluid delivery tube 44 can deliver cooling and irrigating fluid into coaxial fluid channel 38, which allows the fluid to pass distally along the length of the laser energy transmission guide 115 within large guide tube 32, through guide tube transition coupler 34, and into laser guide tube 36. In such embodiments, the guide tube components (large guide tube 32, guide tube transition coupler 34, and laser guide tube 36) can be joined together, to the proximal end cap 26, and to the aspiration cannula 112 outer wall utilizing a means such as soldering or welding.

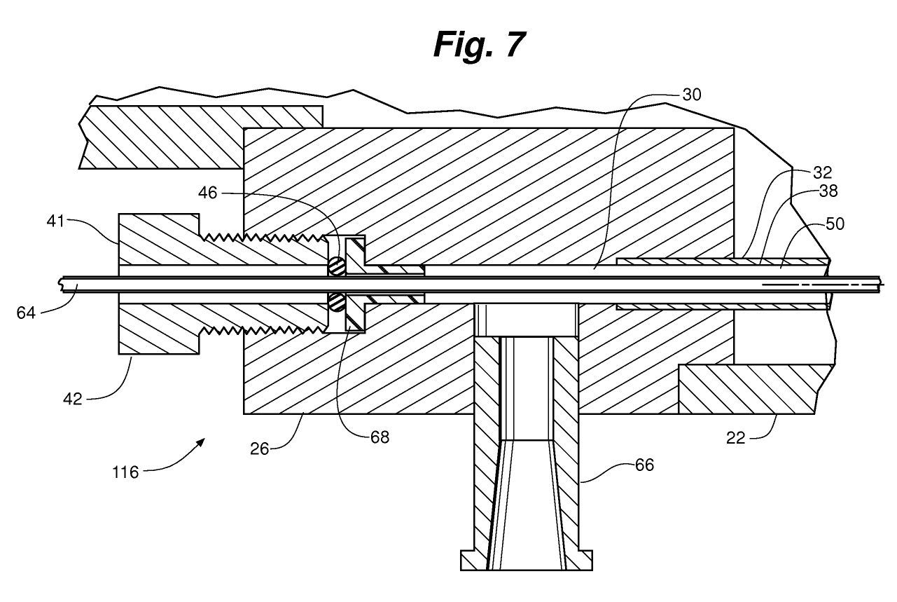

FIG. 7 illustrates minor modifications of another configuration of the present invention which allows the soft tissue aspiration cannula to accommodate an alternative fiber optic delivery system (such as that of FIG. 8) which does not incorporate a Teflon coaxial fluid delivery tube. A bushing 68 is positioned within the fluid and laser guide channel 30 to allow a fluid and air-tight seal at the fluid and energy source port 41. Optional fluid delivery port 66 is provided to allow the passage of cooling and irrigating fluid from a fluid source and pump (not shown) into the coaxial fluid channel 38.

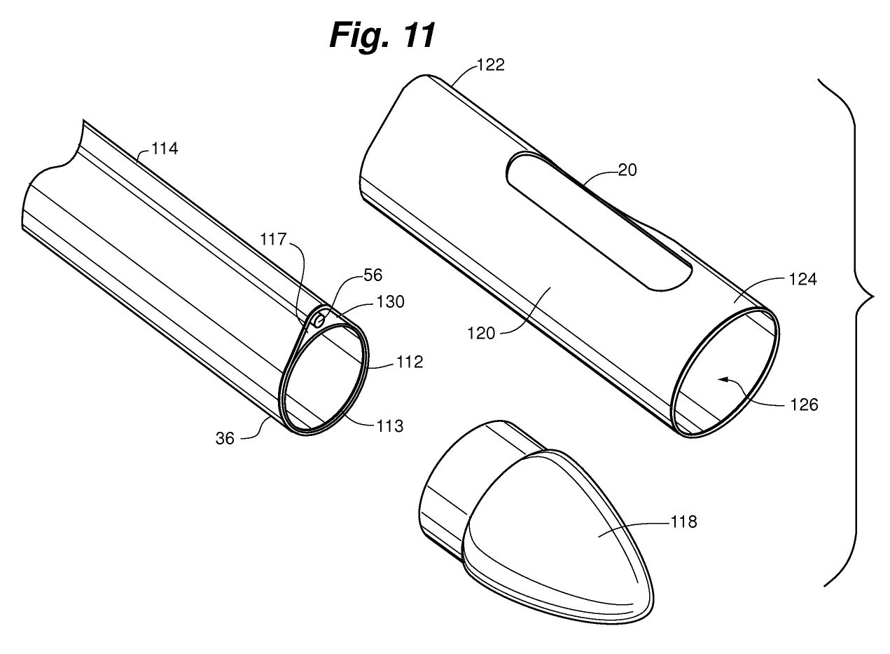

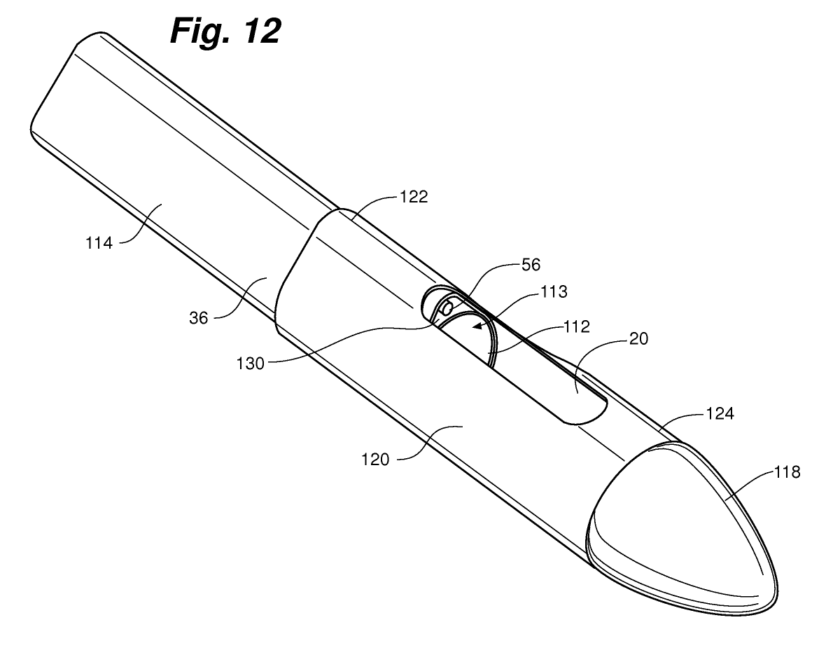

FIGS. 10-14 illustrate another embodiment of a soft tissue aspiration device according to the present invention. FIG. 10 shows a perspective view the distal end 114 of a cannula 112. In this embodiment, both the cannula 112 and laser energy transmission guide 115 are housed within laser guide tube 36. In various embodiments, an oblong cross-sectional shape of the laser guide tube 36 provides a laser guide lumen 117 adjacent the cannula 112. The laser energy transmission guide 115 can extend within the laser guide lumen 117 and in parallel along aspiration cannula 112 to terminal end 56, where laser energy can be dispersed across an aspiration inlet port 20. Some embodiments can include a laser energy diffuser 70 (see e.g. FIG. 10) interposed between fiber end 56, and aspiration inlet port 20 as described above. In various embodiments of the present invention, the diffuser 70 can take the form of a flat window with diffusing surface facing fiber end 56 for preventing direct contact with aspirated tissue, as shown in FIG. 10. In additional embodiments of the present invention, the diffuser can also take the form of a cylindrical section, one end being in contact with fiber end 56, the other end near the aspiration inlet port 20, housed in a protective dielectric sheath in order to preserve its diffusing qualities in the presence of aspirated tissue.

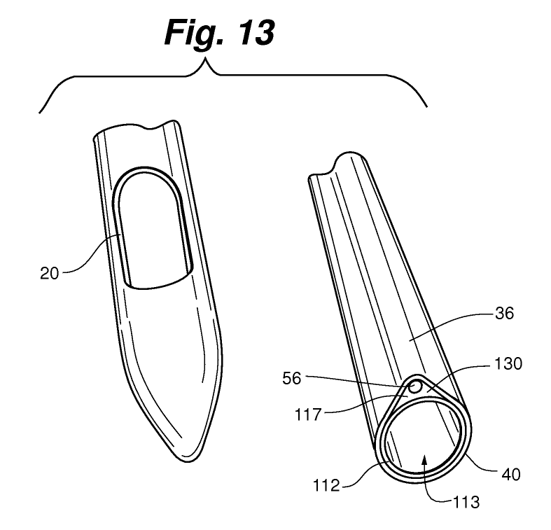

In some embodiments, the aspiration inlet port 20 is located in aspiration inlet cap 120 interposed between laser guide tube 36 and tip 118. Aspiration inlet cap 120 can have a proximal end 122 configured to receive the laser guide tube 36, and a distal end 124 configured to receive the tip 118. The tip 118 can be a disposable tip as described above. Alternatively, aspiration inlet cap 120 can have a tip incorporated into the cap, i.e. the distal end can be sealed and machined to a rounded, bullet or otherwise shaped end (see e.g. FIG. 13). In operation, suction from such embodiments draws soft tissue to be removed through aspiration inlet port 20 into tip cavity 126, which is in fluid communication with lumen 113 via cannula inlet port 128. Laser energy ablates said soft tissue and the ablated tissue can be drawn through cannula inlet port 128, and into lumen 113, where it passes through the cannula 112 and out of the device via a soft tissue outlet port 28 (see e.g. FIG. 1).

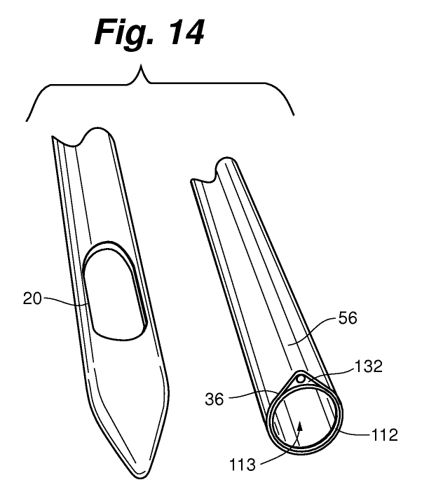

In some embodiments, the laser guide lumen 117 (i.e. the cavity between the outer wall of the cannula 112 and the inner wall of the laser guide tube 36) can leave a crescent-shaped opening 130 located termination point 40 (see e.g. FIG. 13). Such an opening can allow ablated soft tissue or other material to occlude and/or enter the laser guide lumen 117 which can lead to diminished performance, overheating and/or charring of the laser energy transmission guide 115. To prevent this, some embodiments include a means to seal the laser guide lumen. In a various embodiments, a filler material 132, such as an optical epoxy, can be applied at the termination point 40 to seal the crescent-shaped opening 130 (see e.g. FIG. 14) and secure laser energy transmission guide 115.

In some embodiments, filler material 132 can be applied not only at termination point 40, but throughout laser guide lumen 117 along the entire length of the cannula. The filler material 132, such as an epoxy, used in this manner can have other advantages, for example, an epoxy or similar material affixes the laser energy transmission guide within the laser guide lumen, and joins the cannula to the laser guide tube so that the cannula does not move within the outer laser guide tube 36. Moreover, an epoxy or similar material surrounding laser energy transmission guide 115 can act as a heat-sink for the guide 115, thereby eliminating the need for fluid cooling of the guide or fiber. In some embodiments, the filler material 132, such as an epoxy, can include metal or conductive fragments (e.g. aluminum, copper, etc.) dispersed throughout to increase the thermal conductivity of the filler material 132 and better draw heat away from the laser energy transmission guide 115 to prevent charring of the fiber. Alternatively, the laser guide lumen 117 can have conformal fittings (not shown), adapted to receive the laser energy transmission guide 115 and thereby reduce the size of the lumen such that soft tissue material cannot fit within. One embodiment of the present invention uses a high temperature epoxy available, for example, from Thorlabs, Newton, N.J.

Because as discussed above, a filler material 132, such as an epoxy, filling the laser guide lumen 117 can act as a heat sink some embodiments need not use fluid cooled laser energy transmission guides. For example, a guide including a laser fiber sheath 50 and laser fiber 54 (such as that of FIG. 8) can be used. Alternatively, in some embodiments, the laser energy transmission guide can be a laser fiber 54 not having a sheath 50. With such embodiments, a handle similar to that of FIG. 7 as discussed above is appropriate. However in such a handle, because no cooling fluid is introduced to the system, alternative inlet port 66 need not be used so cap 60 can be in place, or the alternative inlet port 66 can be removed.

In various embodiments of the present invention, the handle 22, distal handle end cap 24, proximal handle end cap 26, aspirated soft tissue outlet port 28, fluid and laser fiber large guide tube 32, guide transition coupler 34, laser guide tube 36, aspiration inlet cap 120 and retaining screw 42 are all of stainless steel. However, other suitable materials can also be utilized in manufacturing these components. Also, in some embodiments, the handle 22 can be a molded plastic handle, being contoured to fit a hand. The handle 22 of various embodiments can be of tubing of 1.125″ O.D.×0.125″ wall (1.0″ I.D.) about 3.25″ long. The distal handle end cap 24 in some embodiments is of 1.125″ diameter, machined to fit the handle inside diameter and drilled to accommodate the aspiration cannula outside diameter. In additional embodiments of the present invention, the proximal handle end cap 26 is 1.125″ diameter, machined to fit the handle inside diameter, drilled to accommodate the aspiration outlet port, fluid and laser guide channel, and large guide tube, and drilled and tapped to accommodate the retaining screw. The aspirated soft tissue outlet port 28 in various embodiments is of 0.75″ diameter, machined to fit the proximal handle end cap and tapered to accommodate ⅜″ I.D.×⅝″ O.D. suction tubing, and drilled to a 0.3125″ diameter hole. The fluid and laser fiber large guide tube 32 is 0.120″ O.D.×0.013″ wall (0.094″ I.D.), about 2″ long in various embodiments of the present invention. The guide tube transition coupler 34 is 0.25″ diameter 0.625″ long, drilled to accommodate large guide tube 32 and laser guide 36 in some embodiments of the present invention. In additional embodiments of the present invention, the laser guide tube 36 is of 0.072″ O.D.×0.009″ wall (0.054″ I.D.) in variable lengths, determined by the length of the cannula 112. Retaining screw 42 can be ¼″-28 threads/inch Allen head cap screw 0.75″ long, drilled to accommodate the laser fiber optic delivery system. Also, in some embodiments, plug 60 for fluid source port 66 is a Luer-Lock male plug. Alternative fluid delivery port 66, in various embodiments, is a stainless steel female Luer-Lock. Bushing 68 for laser fiber sheath 50, in some embodiments, is of Teflon 0.120″ O.D.×0.072″ I.D., 0.187″ diameter flange, 0.5″ long, approximate dimension. Also, various embodiments of the present invention can include fluid and laser fiber optic delivery system 62 (suitable for use with embodiments such as those in FIGS. 1-2) available from, for example, Surgical Laser Technologies, Malvern, Pa., Model number: SFE 2.2 and further includes a 2.2 mm (0.086″) outer diameter (“O.D.”) Teflon coaxial fluid delivery tube, 0.8 mm (0.315″) O.D. Teflon laser fiber sheath, and 0.600 mm (0.023″) diameter laser guide fiber length 4.0 meters (157.5″). Another alternative laser (suitable for use with embodiments such as those in FIGS. 10-14) fiber optic delivery system is available from, for example, Heraeus Laser Sonics, Inc., Santa Clara, Calif., model number: B24D and includes a 0.8 mm (0.315″) O.D. Teflon laser fiber sheath, and a 0.600 mm (0.023″) diameter laser guide fiber length 3.66 meters (144″).

In various embodiments of the present invention a laser energy source can be used that generates wavelengths having selective absorption for fat and blood tissue. In some embodiments the light wavelengths can be greater than 800 nm. For example, a laser energy source generating wavelengths from between 800 nm-1000 nm can be used. Additionally, wavelengths ranging from 900 nm-1000 nm can be used. Furthermore, wavelengths ranging from 970 nm-980 nm can be used. Longer wavelengths can also be utilized with embodiments of the present invention (for example wavelengths between 1200 nm-1300 nm, or 1700 nm-1800 nm) as these ranges can also have a high selective absorption for fat tissue.

Additionally, in various embodiments of the present invention the laser energy can be varied during application to direct multiple wavelengths. For example, multiple wavelengths having individual absorption characteristics for blood and fat. Examples of ranges that can be utilized with the devices of the present invention include 532 nm-600 nm and 970 nm-1000 nm, 532 nm-600 nm and 1200 nm-1300 nm, and 532 nm-600 nm and 1700 nm-1800 nm.

Furthermore, the devices of the present invention can further provide pulsed delivery of laser energy. For example, a pulse of laser energy timed with the aspirator suction can provide bursts of higher energy radiation at programmed, intermittent or event activated intervals. In some embodiments, laser sources can be pulsed at different intervals. Various embodiments include laser energy sources operating on duty cycles ranging from 10% to 100%. In one embodiment of the present invention, a laser energy source provides laser energy on a 50% duty cycle.

An example of a laser source for use with an embodiment of this invention using a fluid and laser fiber guide tube system (such as that of FIGS. 1 or 2) is available, for example, from Surgical Laser Technologies, Malvern, Pa., model number SLT CL60, power delivery 0 to 40 watts, with a fluid delivery pump. An alternative laser source for use with embodiments not using a fluid delivery source (such as that of FIGS. 7 and 10) is available, for example from Cooper Laser Sonics, Inc., Santa Clara, Calif., model number: 800, power delivery 0 to 100 watts. While the embodiments discussed above have generally included laser sources, it should be understood that other embodiments may include other energy sources, such as, for example light emitting diodes.

A vacuum aspirator (not shown) for providing suction within the lumen 113 can be of any suitable type, such as that available from Wells Johnson Co., Tucson, Ariz., model: General Aspirator, vacuum 0 to 29+ CFM. The aspirator can be coupled with the outlet port 28 with suction tubing available, for example, from Dean Medical Instruments, Inc. Carson, Calif., at ⅜″ I.D.×⅝″ O.D. in various embodiments of the present invention. A fluid pump (not shown) for delivering a cooling and cleaning lavage via the device, can be of any suitable type, such as an IVAC Volumetric Infusion pump, Model No. 590, available from IVAC Corporation, San Diego, Calif.

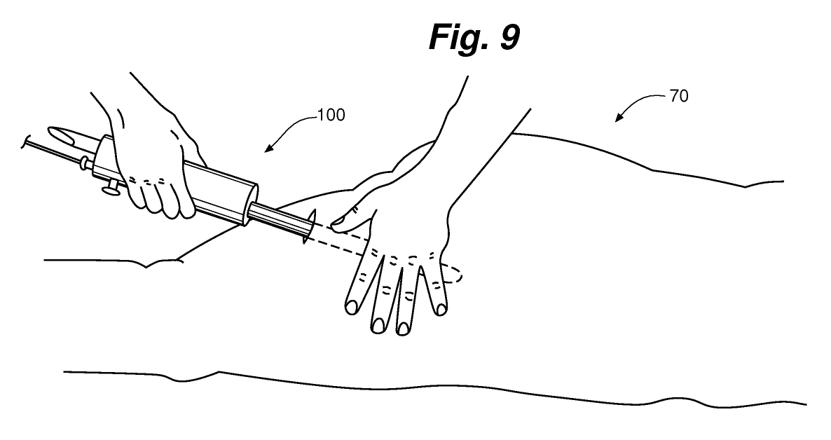

To perform one of the methods of the present invention, as illustrated in FIG. 9, the surgeon determines the location and extent of soft tissue to be removed. The appropriate size laser soft tissue aspiration device 100 is selected. A short incision is made and the cannula tip 118 and the distal end of the cannula 114 is passed into the soft tissue to be removed. In embodiments including a fluid and laser fiber guide tube system (e.g. the embodiments of FIGS. 1 and 2), the fluid delivery pump is activated, delivering normal saline through the Teflon fluid delivery tube 44, into the coaxial fluid channel 38, to the terminal point of the fluid and laser fiber guide tube 40. The application of a fluid flow of normal saline along the fiber to the fiber tip serves to cool the laser fiber 54 and maintain the terminal point of the laser fiber 56 and terminal point of the laser guide tube 40 free of tissue and other detritus. The aspiration pump is then activated. It is noted that the devices of the present invention can include sensors that indicate proper coolant and suction activity and thereby inhibit the activation of the laser fiber by a safety switch if proper coolant or suction are not present. The negative pressure thus generated is transmitted to the laser soft tissue device 100 via a flexible suction tubing, to the soft tissue outlet port 28, through the handle 22, through the cannula 112, to the soft tissue aspiration inlet port 20. The resultant negative pressure at the inlet port draws a small portion of the soft tissue into the lumen 113 of the cannula 112. The laser is then activated. The laser energy is transmitted to the terminal point of the laser fiber 56 and into the soft tissue within the cannula lumen 113, cleaving the soft tissue and coagulating small blood vessels. Additional soft tissue enters the soft tissue inlet port 20 by virtue of a reciprocating longitudinal motion of the laser soft tissue aspiration device 100 within the soft tissue. This reciprocating motion is applied by the surgeon's hand on the handle 22. The reciprocating motion of the laser soft tissue aspiration device, with respect to the surrounding soft tissue, is facilitated by the stabilization of the soft tissue by the surgeon's other hand placed on the skin overlying the cannula soft tissue inlet port 20. Soft tissue is removed from the vicinity of the inlet port 20 to the more proximal portion of the lumen 113 of the cannula, and eventually out the cannula to the soft tissue outlet port 28 by the negative pressure generated by the aspiration pump.

By utilizing the present laser soft tissue aspiration device according to the present method, a variety of advantages are achieved. The ND:YAG laser energy or other fiber delivered laser energy capable of coagulation and cutting will decrease blood loss and render the surgical procedure safer by coagulating small blood vessels in the surgical area. By enabling the cutting of the soft tissue in a straighter line, the scooping, ripping and tearing action characteristic of other devices, will be eliminated, resulting in more precise soft tissue removal, fewer contour irregularities and enhanced patient satisfaction. With the addition of the cutting action of the laser energy provided by the present invention the rate of removal of unwanted soft tissue is greatly enhanced over that of previous devices and techniques thus decreasing operative time. By completely confining the laser energy safely and efficiently within the lumen of the cannula, these benefits are obtained without fear of peripheral laser thermal damage. The fluid flow in some embodiments, in addition to providing cooling and cleaning of the laser fiber, will prevent tissue adherence to and potential damage to the sensitive laser fiber tip. The fluid flow will also assist in solubilizing and emulsifying the fatty tissue serving to further facilitate aspiration and prevent clogging of the cannula throughout the procedure. Moreover, the external positioning of the laser guide tube provides a smooth, undisturbed cannula lumen less susceptible to occlusion from ablated soft tissue material.

Thus, the present invention provides an improved device for use in surgical removal of soft tissue. Animal studies and clinical studies to date utilizing the present invention for surgical body contouring by removing fat have demonstrated less cannula occlusion, less bleeding, less post-operative pain and bruising, excellent cosmetic results, and generally a more aesthetic procedure than has been possible with previous soft tissue aspiration techniques.

While the invention has been illustrated and described in detail in the drawings and foregoing description, such an illustration and description is to be considered as exemplary and not restrictive in character, it being understood that only various embodiments of the present invention have been shown and described and that all changes and modifications that come within the spirit of the invention are desired to be protected.

What is claimed is:

1. A laser soft tissue aspiration device comprising:
an aspiration cannula having a proximal end and a distal end, the aspiration cannula having a lumen provided with fluid flow connection to an aspirated soft tissue outlet port at the proximal end;
at least one aspiration inlet port adjacent to the aspiration cannula distal end and in fluid flow connection to the lumen;
a laser guide tube extending longitudinally along the exterior of the aspiration cannula, said laser guide tube extending from the aspiration cannula proximal end and terminating at a laser guide tube termination point near the at least one aspiration inlet port; and
a laser energy transmission guide extending longitudinally within said laser guide tube and external to the aspiration cannula, said laser energy transmission guide extending from a laser energy source at the aspiration cannula proximal end to a point near the laser guide tube termination point, the laser energy transmission guide being configured to direct laser energy substantially across the aspiration inlet port.

2. The laser soft tissue aspiration device of claim 1, wherein the aspiration cannula is disposed within the laser guide tube, thereby providing a laser guide lumen between an outer diameter of the aspiration cannula and an inner diameter of the laser guide tube, the laser energy transmission guide being disposed within said laser guide lumen.

3. The laser soft tissue aspiration device of claim 2, further comprising:
an aspiration inlet cap, said aspiration inlet cap having adapted to receive the distal end of the laser guide tube and having a cavity in fluid flow connection with the lumen, the aspiration inlet port being disposed within the aspiration inlet cap.

4. The laser soft tissue aspiration device of claim 2, further comprising a filler material disposed at the distal end of the laser guide tube, the filler material configured to seal the laser guide lumen.

5. The laser soft tissue aspiration device of claim 4, wherein the filler material extends throughout the laser guide lumen.

6. The laser soft tissue aspiration device of claim 5, wherein the filler material includes thermally conductive fragments.

7. The laser soft tissue aspiration device of claim 5, wherein the filler material comprises a thermally conductive material.

8. The laser soft tissue aspiration device of claim 4, wherein the filler material comprises an epoxy.

9. The laser soft tissue aspiration device of claim 4, wherein the filler material is configured to diffuse the laser energy directed from the laser energy transmission guide.

10. The laser soft tissue aspiration device of claim 2, wherein the laser guide tube includes conformal fittings adapted to receive the laser energy transmission guide.

11. The laser soft tissue aspiration device of claim 1, wherein the laser guide tube is adapted to accommodate a fluid and laser fiber guide tube system, having a coaxial fluid channel about the laser energy transmission guide, thereby providing for fluid cooling of the laser energy transmission guide.

12. The laser soft tissue aspiration device of claim 1, wherein the laser guide tube termination point intersects the lumen opposite the aspiration inlet port.

13. The laser soft tissue aspiration device of claim 1 wherein the laser guide tube termination point is distal relative to the aspiration inlet port.

14. The laser soft tissue aspiration device of claim 1, further comprising a reflective surface proximate the laser guide tube termination point and configured to reflect the laser energy across aspiration inlet port.

15. The laser soft tissue aspiration device of claim 14, wherein the reflective surface is a flat mirror disposed within the lumen.

16. The laser soft tissue aspiration device of claim 14, wherein the reflective surface is an inner surface of a cannula tip.

17. The laser soft tissue aspiration device of claim 14, wherein the reflective surface is generally parabolic.

18. The laser soft tissue aspiration device of claim 1, further comprising a diffuser or focusing device interposed between the laser energy transmission guide and the aspiration inlet port.

19. The laser soft tissue aspiration device of claim 18, wherein the diffuser or focusing device comprises an optical epoxy.

20. The laser soft tissue aspiration device of claim 18, wherein the diffuser or focusing device is disposed within a cannula tip.

21. The laser soft tissue aspiration device of claim 1, wherein the laser energy transmission guide comprises a laser fiber and a sheath.

22. The laser soft tissue aspiration device of claim 21, wherein the sheath is a Teflon sheath.

23. The laser soft tissue aspiration device of claim 1, further comprising a safety switch, adapted to prevent the laser energy from entering the laser energy transmission guide upon triggering of said safety switch.

24. The laser soft tissue aspiration device of claim 23, further comprising a temperature sensor disposed within the laser guide tube and configured to trigger the safety switch upon overheating of the laser energy transmission guide.

25. The laser soft tissue aspiration device of claim 23, further comprising a pressure sensor disposed within the aspiration cannula and configured to trigger the safety switch upon determination of improper internal pressure within the lumen.

26. An in vivo surgical method of aspirating soft tissue from a patient comprising:
inserting an aspiration cannula through the patient's epidermis, so that a distal end of the aspiration cannula is positioned in an area of soft tissue, said aspiration cannula provided with a lumen in fluid flow communication with at least one aspiration inlet port adjacent the aspiration cannula distal end;
providing laser energy from a laser energy source to a laser energy transmission guide extending longitudinally within a laser guide tube, said laser guide tube extending longitudinally along the exterior of the aspiration cannula, the laser energy transmission guide transmitting the laser energy to a point near the at least one aspiration inlet port and being configured to direct laser energy substantially across the aspiration inlet port to perform localized soft tissue cutting and blood vessel coagulation;
providing an aspiration source at a proximal end of said aspiration cannula to aspirate soft tissue through said aspiration inlet port and said aspiration cannula;
activating the aspiration source; and
activating the laser energy source.

27. The method of claim 26, further comprising the steps of:
providing a temperature sensor within the laser guide tube;
determining a temperature reading of the laser energy transmission guide via the temperature sensor; and
inhibiting the activation of the laser energy source if the temperature reading registers a predetermined improper reading.

28. The method of claim 26, further comprising the steps of:
providing a pressure sensor within the lumen;
determining a pressure reading from within the lumen via the pressure sensor; and
inhibiting the activation of the laser energy source if the pressure reading registers a predetermined improper reading.
